(19) United States
(12) Patent Application Publication
(10) Pub. No.: US 2016/0345815 A1
(43) Pub. Date: Dec. 1, 2016

(54) Dental Mirror

(71) Applicant: Leon Chen, Taipei City (TW)

(72) Inventors: Leon Chen, Taipei City (TW); Nina Chen, Las Vegas, NV (US); Audree Chen, Las Vegas, NV (US); Nick Chen, Las Vegas, NV (US); Alec Chen, Las Vegas, NV (US); Nasdaq Chen, Las Vegas, NV (US); Jennifer Cha, Las Vegas, NV (US)

(21) Appl. No.: 15/169,931

(22) Filed: Jun. 1, 2016

(30) Foreign Application Priority Data
Jun. 1, 2015 (CN) 201510292648.5

(51) Int. Cl. A61B 1/247; A61C 17/06

(52) U.S. Cl. CPC A61B 1/247; A61C 17/043

(57) ABSTRACT

A dental mirror includes a handle, a mirror arranged at one end of the handle, and a pipe connected to the handle and extended beyond the mirror. The pipe is partially formed with a plurality of liquid-sucking holes. When dentist uses the dental mirror, it can suck up liquid or debris in oral cavity of the patient.

### BACKGROUND OF THE INVENTION

1. Field of the Invention

The present invention is related to a dental mirror. In particular, the present invention relates to a dental mirror intended for use in dental and surgical operations so that a dentist can observe patient's oral cavity or teeth conditions.

2. Description of Related Art

In general, no matter during dental operation or dental examination, the oral cavity of dental patient usually has water, saliva, or cooling liquid (usually is clean water) when drilling teeth. To smooth the dental operation or dental examination, such water, saliva, or cooling liquid all need dental machine or tool to extract out or exhaust, so as to avoid affecting a working vision of dentist or dental assist.

As shown in FIG. 1, a drilling equipment 7 and a dental mirror 8 are usually used during dental treatment, such as caries restoration, scaling, and other treatments for patient's teeth. The drilling equipment 7 can be used to clean calculus, drill alveolar bone or process dental operation. The dental mirror 8 can be moved or rotated in the oral cavity M, so that the dentist or dental assistant can observe the condition in the oral cavity M by the dental mirror 8. In this figure, the dental mirror 8 is used to keep patient's tongue or cheek away from the teeth of treating, and reflect the treating area by outer light for dentist. The dental mirror 8 can help dentist to observe the gingival tissue that can't be seen by looking straightly.

Moreover, the patient will produce water during dental examination, or dental treatment; and clean water is also used to cool the alveolar bone when drilling the alveolar bone. Thus, a suction device 9 is used to extract out the washing liquid sprayed by the drilling equipment 7, patient's saliva, and tooth fragment. A long tube is only shown in this figure to represent the suction device 9. In fact, one end of the suction device 9 is connected with a motor (not shown), so that the suction device 9 can continuously process an extraction.

The drilling equipment 7, the dental mirror 8, and the suction device 9 can't be simultaneously held and operated by one single. Therefore, the dentist usually needs a dental assist for assistance. The dentist operates the drilling equipment 7 and the dental mirror 8, and dental nurse operates the suction device 9. The dental nurse's task is to move the suction device 9 to suck the liquid from the drilling equipment 7 flowing to the dental mirror 8, so as to ensure the cleanness of the dental mirror 8 during dental treatment.

If some condition or the dental nurse is absent, the dentist needs to move the suction device 9, and the dental mirror 8 is put aside. In the meantime, the patient has to be arranged in a special posture, so that the dentist can clearly observe the treating area. However, the dentist sometimes needs to work in some postures very uncomfortably. Inevitably, the dentist has to interrupt therapy and again uses the dental mirror 8 to see the treating area. Then, to move the suction device 9 to another position to suck liquid. The alternate using of the dental mirror 8 and the suction device 9 causes therapy many pauses, and the therapy process take up time longer than the required time.

In addition, numerous dental tools arranged in a narrow oral cavity M will obstruct the dental's checkups, treatment, or surgery, which is not ideal.

Therefore, it is a goal that a person having ordinary knowledge in the art to reach is, how to integrate and simplify the dental tools so that the dentist can more conveniently to process dental checkups, treatment or surgery procedures, and use and operate the dental mirror conveniently by oneself.

### SUMMARY OF THE INVENTION

It is one objective of this invention to provide a dental tool, which combines a dental mirror and liquid sucking structure, so that when dentist is using the dental mirror, the liquid or fragment in patient's oral cavity can be sucked simultaneously to conveniently process dental consultation, treatment or surgical operations.

It is another objective of this invention to provide a dental tool for a dentist can operate independently by oneself, so as to reduce tools in oral cavity with convenience and space saving.

In order to achieve the above objectives, the present invention is to provide a dental mirror, which includes a handle, a mirror unit and a tube. The mirror unit is disposed at one end of the handle. The tube is arranged on a periphery of the mirror unit, and the tube is formed with a plurality of inhaling holes.

According to one embodiment of the dental mirror, the tube is detachably connected to the handle.

According to one embodiment of the dental mirror, the tube has a free end a fixing end, the fixing end is fixedly arranged on or detachably connected to the handle, a plurality of inhaling holes are formed between the free end and the fixing end.

According to one embodiment of the dental mirror, the tube includes at least two fork portions, and the at least one fork portion is formed with several inhaling holes.

According to one embodiment of the dental mirror, the handle is hollow-shaped, an inner portion of the tube is connected with an inner portion of the handle.

According to one embodiment of the dental mirror, the handle is hollow-shaped, and the tube is extended and disposed in the handle.

According to one embodiment of the dental mirror, the handle is hollow-shaped, and the tube is inserted in the handle.

According to one embodiment of the dental mirror, the tube is fixed to a periphery of the handle.

According to one embodiment of the dental mirror, one end of the tube has a connecting element, and the connecting element includes an inserting portion and a blocking portion connected to the inserting portion. The inserting portion is inserted in the handle, and the blocking portion is abutted against an outer edge of the handle. In one further embodiment, the inserting portion has at least one hook formed on a periphery thereof, and an inner of the handle is formed with at least one groove, so that the hook and the groove can be engaged mutually.

According to one embodiment of the dental mirror, the tube is extended from the handle and attached to a back surface of the mirror unit. In one further embodiment, the inhaling holes of the tube are distributed on one-third length of the mirror unit along a longitudinal direction of the handle.

According to one embodiment of the dental mirror, the tube is surrounded in an annular manner and attached to an edge of a back of the mirror unit.

According to one embodiment of the dental mirror, the inhaling holes are distributed through a whole length of the tube.

According to one embodiment of the dental mirror, a cross-sectional shape of the tube is flat-shaped and attached to a back of the mirror unit. In one further embodiment, the inhaling holes are formed on a back of the mirror unit, and the tube has an opening of arc-shaped, the arc-shaped opening is trimmed to a periphery of the mirror unit.

According to one embodiment of the dental mirror, the mirror unit includes a cover and a glass component disposed on the cover, and the tube has a supporting part extended to the glass component. The supporting part is abutted against the glass component.

According to one embodiment of the dental mirror, an end of the tube has an opening, and the opening is passed through the mirror unit.

Thus, the present invention has advantages as follows. When the dentist uses the dental mirror of this present invention, liquid or fragment in the patient's oral cavity can be sucked, and does not alternatively use a traditional dental mirror and suction device. Many pauses during therapy process can be avoided, and time of therapy process is saved. Manpower of dental assist is reduced effectively. In addition, the dental mirror of the present invention can be operated by dentist oneself single, and is not occupied the space of the oral cavity, which is used more conveniently.

For further understanding of the present invention, reference is made to the following detailed description illustrating the embodiments and examples of the present invention. The description is for illustrative purpose only and is not intended to limit the scope of the claim.

### BRIEF DESCRIPTION OF THE DRAWINGS

FIG. 1 is a perspective view of a dental mirror of prior art used with a drilling equipment and a suction device.

FIG. 2A is a side view of dental mirror of first embodiment according to the present invention.

FIG. 2B is a perspective view of the dental mirror of first embodiment according to the present invention.

FIG. 3 is a perspective view of the dental mirror being used in an oral cavity according to the present invention.

FIG. 4 is a side view of dental mirror of second embodiment according to the present invention.

FIG. 5 is a side view of dental mirror of third embodiment according to the present invention.

FIG. 6 is a perspective view of dental mirror of fourth embodiment according to the present invention.

FIG. 7 is a perspective view of dental mirror of fifth embodiment according to the present invention.

FIG. 8 is a cross-sectional view showing a first way to connect a tube and a handle of the dental mirror according to the present invention.

FIG. 9 is a cross-sectional view showing a second way to connect a tube and a handle of the dental mirror according to the present invention.

FIG. 10 is a cross-sectional view showing a third way to connect a tube and a handle of the dental mirror according to the present invention.

FIG. 11 is a cross-sectional view showing a fourth way to connect a tube and a handle of the dental mirror according to the present invention.

FIG. 12 is a side view of dental mirror of sixth embodiment according to the present invention.

FIG. 13 is a side view of dental mirror of seventh embodiment according to the present invention.

FIG. 14 is a bottom view of dental mirror of eighth embodiment according to the present invention.

FIG. 15 is a bottom view of dental mirror of ninth embodiment according to the present invention.

FIG. 16 is a perspective view of dental mirror of tenth embodiment according to the present invention.

FIG. 17 is a cross-sectional view of dental mirror of tenth embodiment according to the present invention.

FIG. 18 is a perspective view of dental mirror of eleventh embodiment according to the present invention.

FIG. 19 is a cross-sectional view of dental mirror of eleventh embodiment according to the present invention.

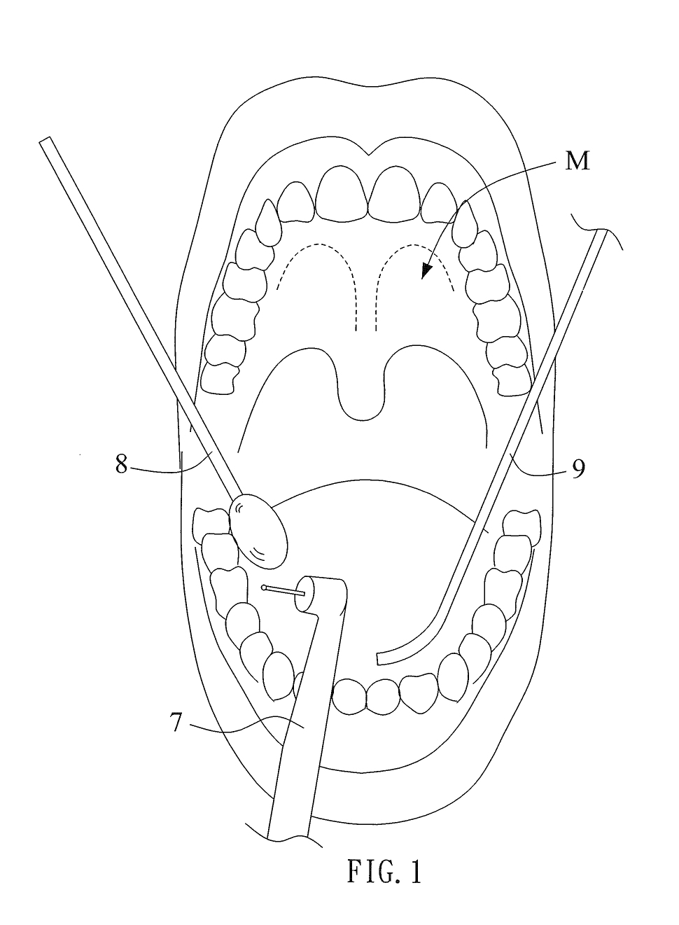

### DETAILED DESCRIPTION OF THE PREFERRED EMBODIMENTS

The aforementioned illustrations and following detailed descriptions are exemplary for the purpose of further explaining the scope of the present invention. Other objectives and advantages related to the present invention will be illustrated in the subsequent descriptions and appended drawings.

### First Embodiment

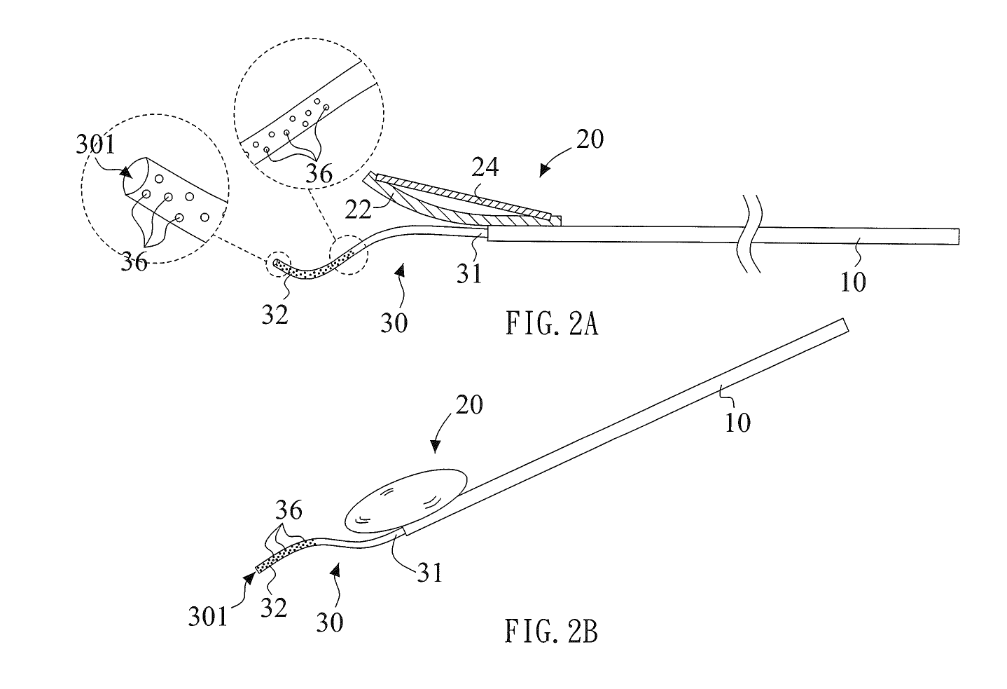

Please refer to FIGS. 2A and 2B, which is a side view and perspective view of dental mirror of first embodiment of the present invention. The present invention provides a dental mirror, or called mouth mirror, which includes a handle 10, a mirror unit 20 connected to one end of the handle 10 and a tube 30. The handle 10 can be made of metal material, plastic material or other material . . . etc. The mirror unit 20 includes a cover 22, and a glass component 24 disposed on the cover 22. The glass component 24 is detachably engaged with, adhered to or clipped on the cover 22. The tube 30 is connected to the handle 10 and extended to a back of the mirror unit 20. A part of the tube 30 is formed with a plurality of inhaling holes 36 for sucking liquid. In other embodiments, the tube 30 can be arranged on a periphery of the mirror unit 20, and the mirror unit 20 can be disposed at one left end of the tube 30.

In this embodiment, the handle 10 can be hollow-shaped. The tube 30 includes a fixing end 31 and a free end 32. The free end 32 is disposed at left side, and the fixing end 31 is disposed at right side. The fixing end 31 is fixedly connected to a left end of the handle 10, and the free end 32 is formed with an opening 301.

As shown in an enlarged view of FIG. 2A and FIG. 2B, the location of the inhaling holes 36, is approximated to the free end 32 of the tube 30, which is at the end of the tube 30. The inhaling holes 36 are distributed on an entire length of the tube 30, and have a width even larger than a width of one tooth, or have a width equal to a width of three or four teeth.

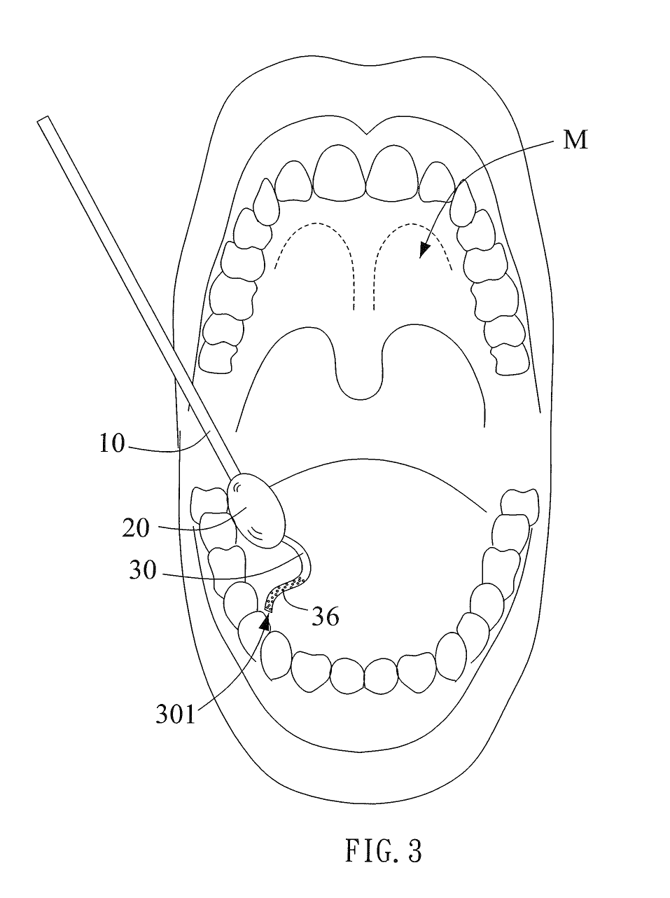

As shown in FIG. 3, that is a perspective view of the dental mirror being used in an oral cavity according to the present invention. When a dentist operates the dental mirror of this present disclosure, the mirror unit 20 of the dental mirror can be used to observe the condition in the oral cavity M, in the meantime, the tube 30 can be used to suck out the water, liquid or fragment in patient's oral cavity M. In this embodiment, a motor (not shown) can be connected to an end of the handle 10 of the dental mirror for sucking water or liquid. When the motor is turned on, the sucking force can be transferred through the handle 10, the tube 30, the inhaling holes 36 and opening 301 of the dental mirror in order, to suck water and liquid. The water and liquid which are sucked out can be exhausted through the tube 30, and handle 10. Refer to FIG. 2A, the opening 301 of the free end 32 has a diameter substantially equal to a diameter of the tube 30, and a diameter of the inhaling holes 36 is smaller than a diameter of the opening 301. Thus, after the motor is turned on, a great quantity of water and liquid can be sucked through the opening 301 of the free end 32. If the opening 301 is blocked by food debris, it still can continuously suck water through the inhaling holes 36 on the tube 30, and the suction process will not be interrupted. In addition, the tube 30 can be made of soft material, or flexible material (such as rubber, plastic). Therefore, the free end 32 can be curved or bent, and is disposed in an area accumulated much watery fluid in the oral cavity M, and the opening 301 and the inhaling holes 36 are conveniently used for sucking water. Through whole process, the dentist can operate just by himself single very conveniently, and does not rely on assisting of the dental assist, temporarily stop treating or change hand. In addition, the tube 30 and the mirror unit 20 can be combined in a separable manner, or an integral structure. The tube 30 can extended along a direction away from the handle 10 and has a length beyond the mirror unit 20. In other word, the tube 30 is longer than the handle. Thus, the tube 30 can be changed its shape or bent more arbitrarily, to be disposed any position in the oral cavity M for sucking water and liquid. For example, the tube 30 can be extended and disposed between teeth and tongue.

### Second Embodiment

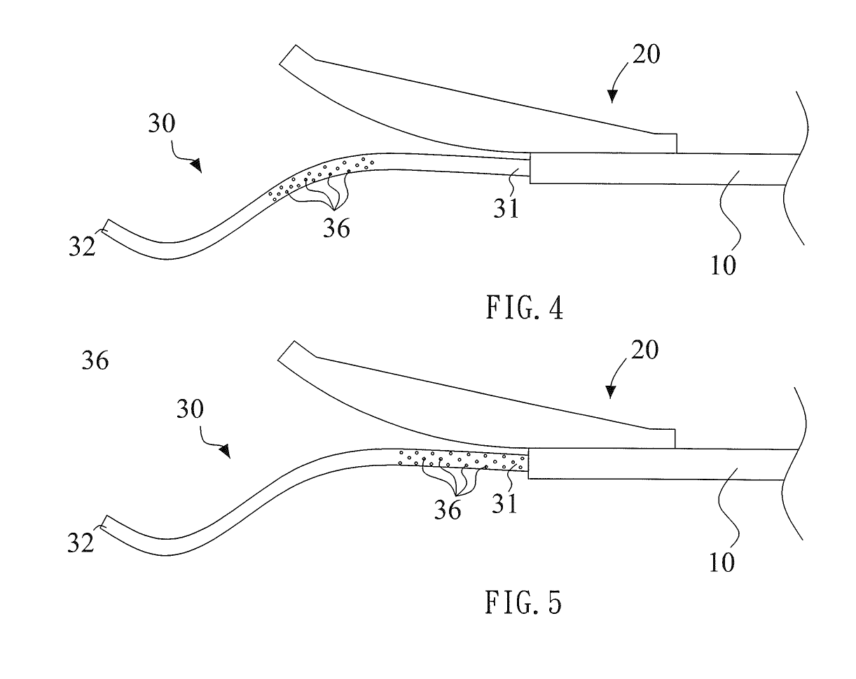

Please refer to FIG. 4, which is a side view of dental mirror of second embodiment according to the present invention. Different from the above embodiment, this embodiment has a plurality of inhaling holes 36, which are formed away from a free end 32 of the tube 30 slightly. For this embodiment, the inhaling holes 36 are distributed on a middle part of the tube 30 which is exposed outside the handle 10, that is, between the fixing end 31 and the free end 32.

### Third Embodiment

Please refer to FIG. 5, which is a side view of dental mirror of third embodiment according to the present invention. Different from the above embodiment, the inhaling holes 36 of this embodiment are formed away from the free end 32 of the tube 30, and closed to the handle 10, that is closed to fixing end 31 of the tube 30.

### Fourth Embodiment

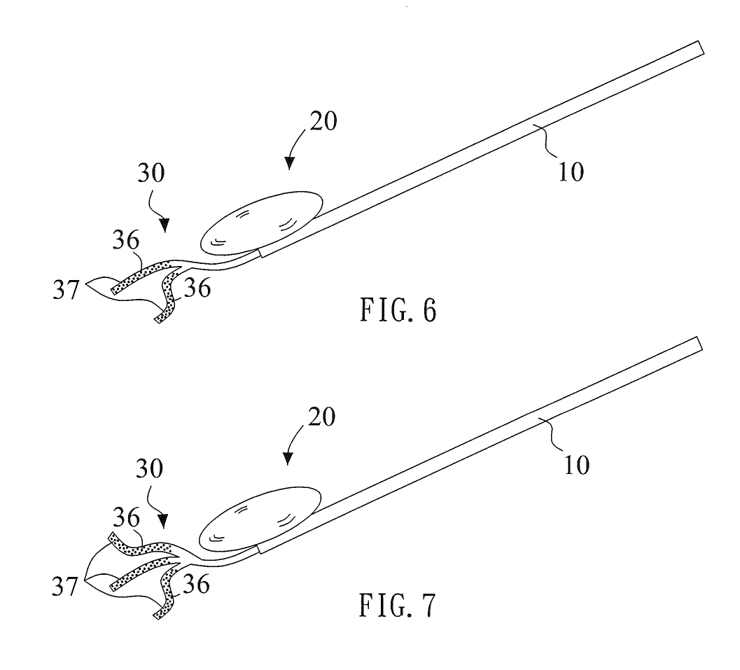

Please refer to FIG. 6, which is a perspective view of dental mirror of fourth embodiment according to the present invention. The tube 30 of this embodiment has an end shaped in a fork, and includes two fork portions 37. Each fork portion 37 is formed with a plurality of inhaling holes 36. The two fork portions 37 have advantage that can suck water or liquid at different positions in oral cavity M. For example, two fork portions 37 can be disposed at a left side and a right side of tongue respectively. Accordingly, no matter how is the liquid flowing in oral cavity, the two fork portions 37 can properly suck liquid.

### Fifth Embodiment

Please refer to FIG. 7, which is a perspective view of dental mirror of fifth embodiment according to the present invention. The tube 30 of this embodiment is fork-shaped, and has three fork portions 37. Each fork portion 37 is formed with a plurality of inhaling holes 36. The three fork portions 37 can arranged at two sides of tongue and underneath a front of tongue, respectively, so as to more thoroughly suck liquid in oral cavity.

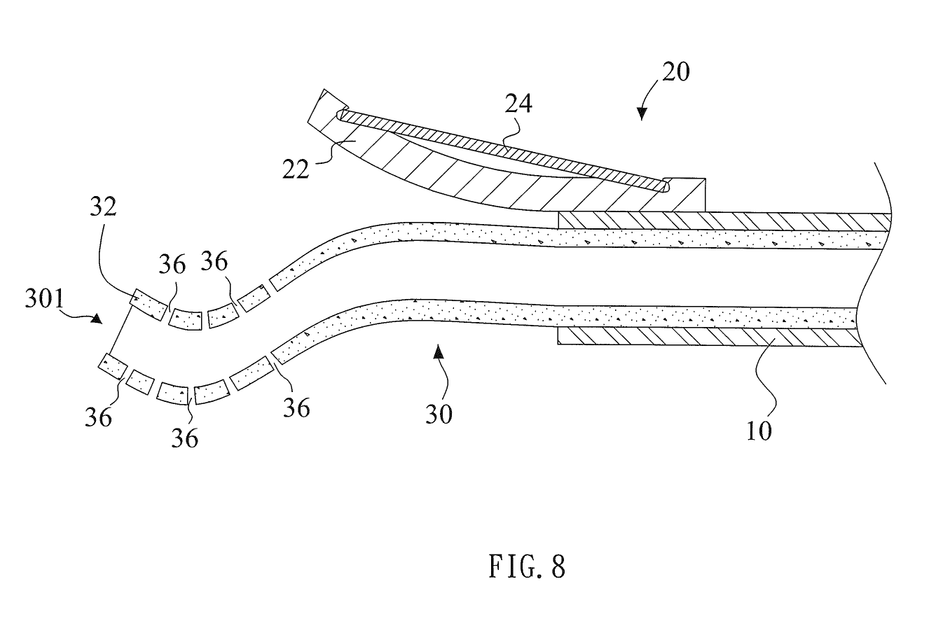

Further, the present invention also provides many ways to connect the tube 30 and the handle 10 of mouth mirror. As shown in FIG. 8, which is a cross-sectional view showing a first way to connect a tube and a handle of the dental mirror according to the present invention. The handle 10 of this embodiment is hollow-shaped, the tube 30 is extended passing through or disposed on an inner wall of the handle 10. However, in other embodiments, the tube 30 of the present invention can be fixed on a periphery of the handle 10, or is twined side by side (not shown).

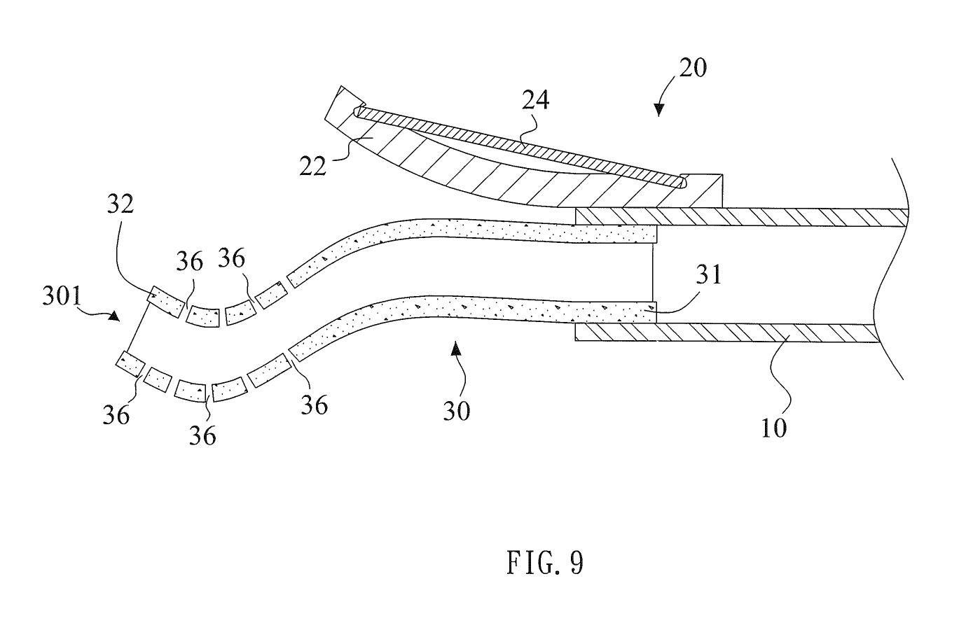

Further, as shown in FIG. 9, which is a cross-sectional view showing a second way to connect a tube and a handle of the dental mirror according to the present invention. In this embodiment of FIG. 9, handle 10 is hollow-shaped. The fixing end 31 of the tube 30 is slightly extended into the handle 10, so that the tube 30 and the handle 10 are communicated mutually and internally. Accordingly, the mutual communication of the tube 30 and the handle 10 has objectives that, water or liquid, which are sucked by the opening 301 or the inhaling holes 36 at left end of the tube 30, can be exhausted toward the right side of the dental mirror. Besides, the tube 30 has only one small section connected to the handle 10, or disposed in the handle 10. In other embodiment, the tube 30 can be fixed or covered on an outer side of the handle 10 (not shown). The tube 30 has an outer diameter substantially equal an inner diameter of the inner passage of the handle 10, and is smaller than an outer diameter of the handle 10. As a result, the tube 30 can be inserted in an inner tube wall of the handle 10, and jointed with the handle 10 mutually in a tight-fit or adhesive manner. Of course, the fixing end 31 of the tube 30 can be detached from or attach with the handle 10 according to demand. In addition, the cover 22 of the mirror unit 20 is fixed on a periphery of the handle 10. The inhaling holes 36 are distributed on one end of the tube 30, and closed to the free end 32.

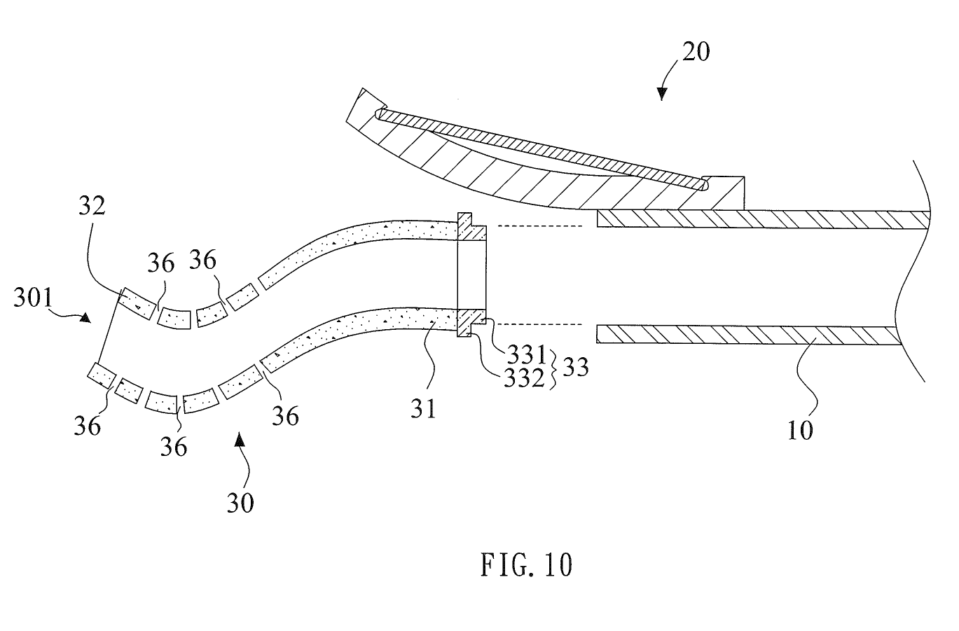

Refer to FIG. 10, which is a cross-sectional view showing a third way to connect a tube and a handle of the dental mirror according to the present invention. The tube 30 of this embodiment has one end formed with a connecting element 33. The connecting element 33 has an inserting portion 331 and a blocking portion 332 connected the inserting portion 331. The inserting portion 331 is inserted in or fixed to an inner wall of the handle 10. The blocking portion 332 is abutted against an outer edge of the handle 10. This embodiment has advantage that, the connecting element 33 can be more conveniently dismounted from the handle 10, or assembled with one end of the handle 10. Thus, the tube 30 can be replaceable or disposable, and is replaced with one new tube 30 according to demand whenever necessary. When the tube 30 is consumed, it just needs to pull away the old one, and replaces one new tube 30. When the connecting element 33 is inserted into the left end of the handle 10, inner passages of the tube 30 and the handle 10 are communicated mutually. Thus, water and liquid sucked out from the oral cavity M can be exhausted right hand. The connecting element 33 can be made of a material which is harder than the tube 30, such as rigid plastic. The connecting element 33 and the tube 30 can be combined by adhesive manner or plastic double injection manner.

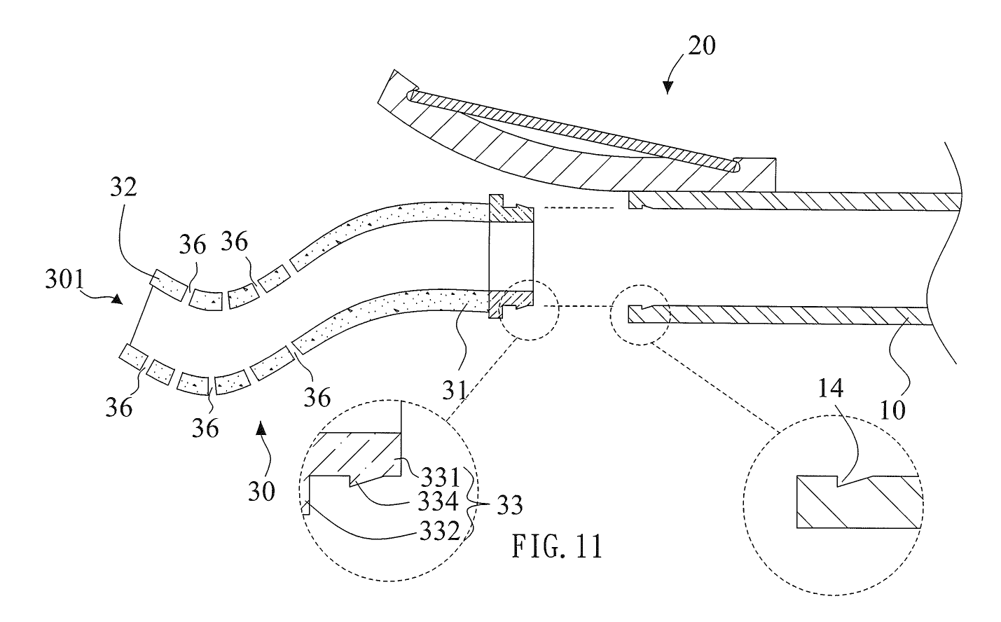

Refer to FIG. 11, which is a cross-sectional view showing a fourth way to connect a tube and a handle of the dental mirror according to the present invention. In this embodiment, different from FIG. 10, the inserting portion 331 further has at least one hook 334 on a periphery thereof. The hook 334 can be ring-shaped and protruded from an outer surface of the inserting portion 331, or be many parts protruded from an outer surface of the inserting portion 331. Correspondingly, the handle 10 is formed with at least one groove 14 on an inner side thereof. The groove 14 can be ring-shaped and concavely formed on an inner wall of the handle 10, or many concaved parts formed on an inner wall of the handle 10. The hook 334 can be selectively engaged with the groove 14, so that the inserting portion 331 can be engaged with or separated from the handle 10 as one please. In this embodiment, the tube 30 not only is replaceable or disposable, but also provides an operative clip feeling when the tube 30 is engaged with the handle 10. The hook 334 can provides a wedged clip feeling during engagement, so that the user can ensure that if the tube 30 is fastened firmly with the handle 10.

A supplementary note, the assembling ways of the tube 30 and the handle 10 in FIG. 8 to FIG. 11 can be also applied in the embodiments in FIG. 2A to FIG. 7 of the present invention.

### Sixth Embodiment

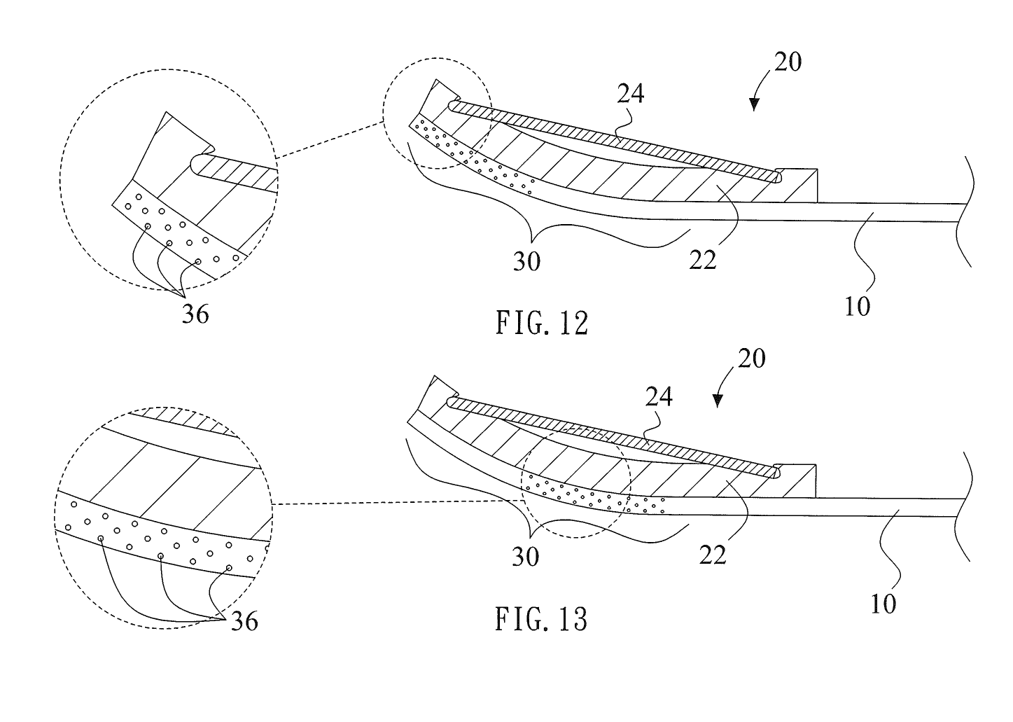

Please refer to FIG. 12, which is a side view of dental mirror of sixth embodiment according to the present invention. In this embodiment, the tube 30 and the handle 10 are integrally formed in one piece and extended to attach to a back of the mirror unit 20. More specifically, the tube 30 is attached to the back surface of the cover 22, so that the inhaling holes 36 of the tube 30 and the inhaling holes 36 closed to the free end of the handle 10. From another viewpoint, the inhaling holes 36 are distributed on an area which has a length about one-third of the mirror unit 20 along a longitudinal direction of the handle 10.

In this embodiment, the handle 10 can be made of metal material or plastic material. The advantages of this embodiment are that, when the tube 30 is used as a water-sucking section and the dentist change the reflecting angle of the mirror unit 20 during operating the mouth mirror, the tube 30 will not swayed due to the rotation of the mirror unit 20. The section for sucking water of the tube 30 is always kept right behind the mirror unit 20.

### Seventh Embodiment

Please refer to FIG. 13, which is a side view of dental mirror of seventh embodiment according to the present invention. One difference between this embodiment and the above embodiment is that positions of the inhaling holes are different. The inhaling holes 36 of this embodiment are formed on a back of the mirror unit 20, which is a middle position of the cover 22. The distributing area of the inhaling holes 36 has a length about equal to one-third length of the mirror unit 20 along a longitudinal direction of the handle 10.

In addition, all embodiments in FIG. 2A to FIG. 13, the inhaling holes 36 are distributed on a part or a local position of the tube 30. But, in other embodiments, the inhaling holes 36 also can be distributed on the whole length of the tube 30. That is, all of the entire tube 30 has inhaling holes 36 entirely distributed thereon.

### Eighth Embodiment

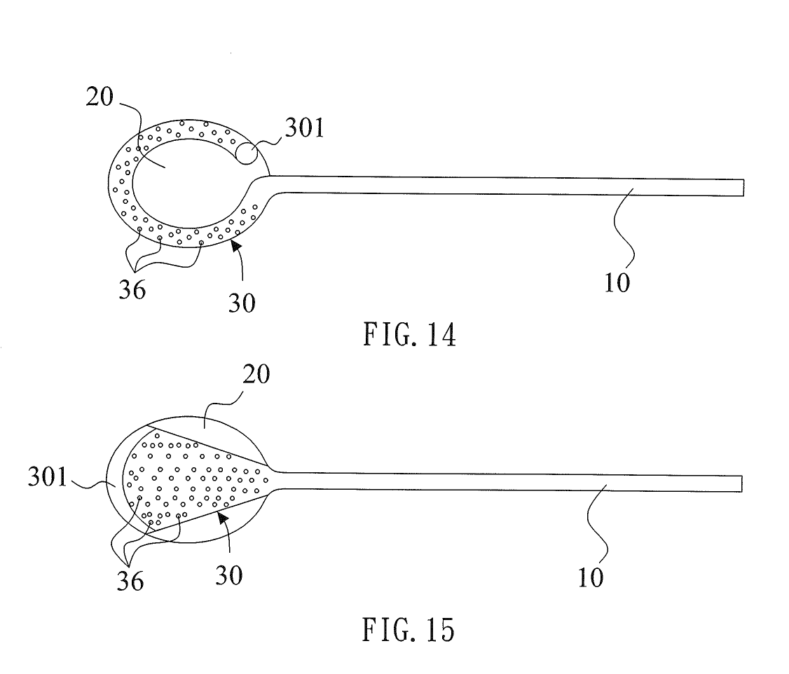

Please refer to FIG. 14, which is a bottom view of dental mirror of eighth embodiment according to the present invention. The tube 30 of this embodiment is also integrally extended from the handle 10 and attached to the back of the mirror unit 20. The difference of this embodiment is that, the tube 30 is encircled annually and attached to an edge of the back of the mirror unit 20. Namely, a length of the tube 30 is substantially equal to a circumference of the mirror unit 20. In addition, the plurality of inhaling holes 36 are distributed on an entire length of the tube 30, so that all circumferential edge of the mirror unit 20 has inhaling holes 36. By using the tube 30 and the inhaling holes 36 on the back of the mirror unit 20, the water or liquid in oral cavity M can be sucked. The free end 32 of the tube 30 is formed with an opening 301, and is bent adjacent to the handle 10. This embodiment has advantages that, no matter how the dental mirror is rotated, the position for sucking water is not limited. There are some inhaling holes 36 always closed to a water-accumulated position closed to gingiva.

### Ninth Embodiment

Please refer to FIG. 15, which is a bottom view of dental mirror of ninth embodiment according to the present invention. In this embodiment, the tube 30 is also integrally extended from the handle 10 and attached to the back of the mirror unit 20. The difference of this embodiment is that, the tube 30 is fan-shaped from a top view. From a cross-sectional view, the tube 30 is flat-shaped and attached to the back of the mirror unit 20. The fan-shaped tube 30 has a plurality of inhaling holes 36 spread thereon, and has an arc-shaped opening 301 formed at a leftmost end thereof. The arc-shaped opening 301 is substantially flushed with a partial circumference of the mirror unit 20, and is far away from the handle 10. This embodiment has advantages that, no matter how is the dental mirror rotated, the position for sucking water is not limited. Even, in some special condition, if great amount liquid needs to be exhausted, the dental mirror can be erected in the oral cavity M. Namely, the bottom edge of the mirror unit 20 is directly faced liquid, and the arc-shaped opening 301 directly faces the position needed to suck liquid. The opening 301 is bigger and can suck liquid more quickly.

### Tenth Embodiment

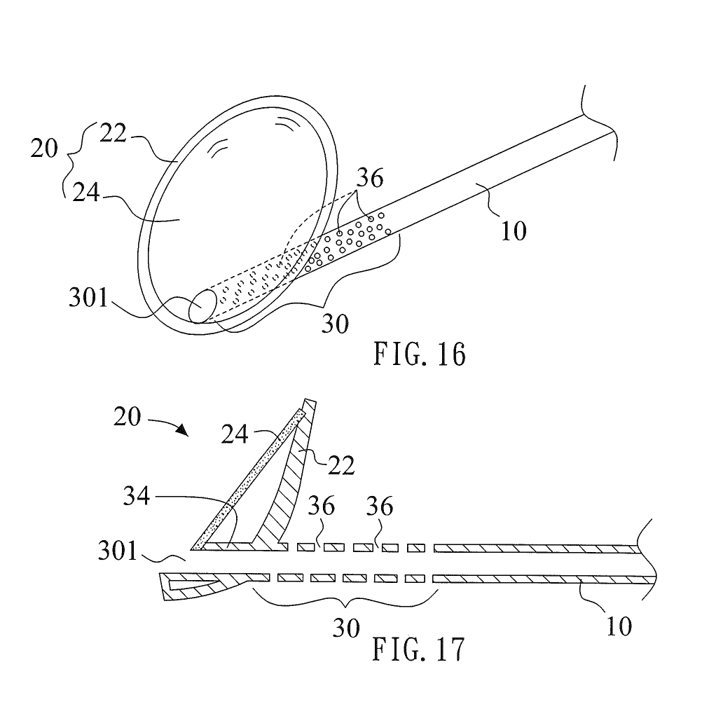

Refer to FIG. 16 and FIG. 17. FIG. 16 is a perspective view of dental mirror of tenth embodiment according to the present invention. FIG. 17 is a cross-sectional view of dental mirror of tenth embodiment according to the present invention. As shown in these Figs of this embodiment, the tube 30 at one end of the dental mirror has an opening 301. The opening 301 is penetrated through the mirror unit 20. In detail, the tube 30 has a left end connected and penetrated through the cover 22 and the glass component 24 of the mirror unit 20, so that water or liquid around the opening 301 can rightward flow through the mirror unit 20, and flows in the tube 30 rightward. In addition, because the tube 30 and the handle 10 are interconnected mutually, water or liquid flowed in the tube 30 can rightward flow through an inner passage of the handle 10. The tube 30 is formed with a plurality of inhaling holes 36. During the dental mirror is used in patient's oral cavity, if the opening 301 is blocked by food residue, the inhaling holes 36 can make certain that the sucking action for water or liquid is still processed. In addition, the tube 30 and the cover 22 of the mirror unit 20 can be an integrally formed structure, or can be two separable and detachable structures. In this embodiment, the tube 30 is extended toward the glass component 24 and has a supporting part 34 between the glass component 24 and the cover 22. The supporting part 34 connected to the glass component 24, and the glass component 24 therefore can endure a larger pressure or impact, to avoid the glass component 24 from breaking because of an excessive force acted thereon. Further, the tube 30, the opening 301 are penetrated through a lower edge of the mirror unit 20.

### Eleventh Embodiment

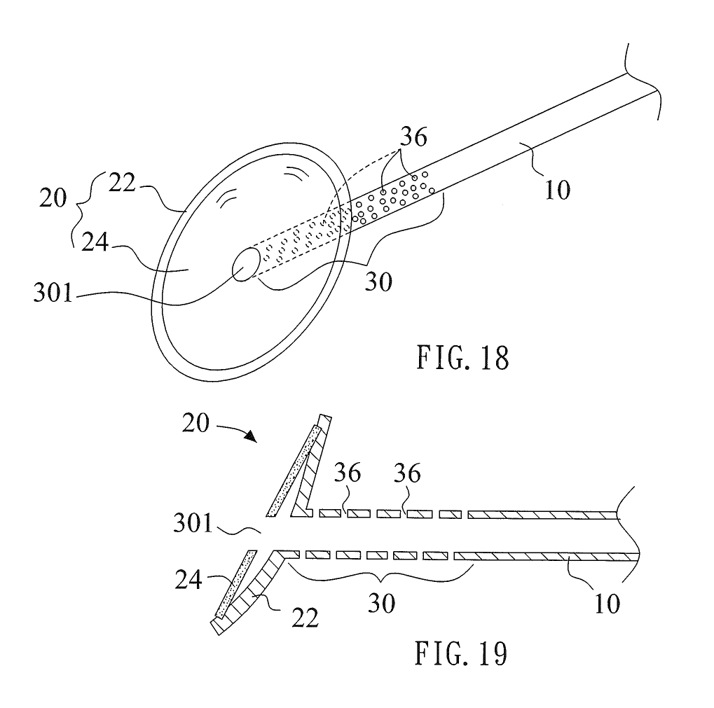

Refer to FIG. 18 and FIG. 19. FIG. 18 is a perspective view of dental mirror of eleventh embodiment according to the present invention. FIG. 19 is a cross-sectional view of dental mirror of eleventh embodiment according to the present invention. As shown in FIG. 18 to FIG. 19, this embodiment is similar to the tenth embodiment in FIG. 16 and FIG. 17, and the most structure is not described redundantly. Different from the above-mentioned structure, this embodiment has no supporting part 34 contacted with the glass component 24. Besides, the tube 30 and opening 301 are penetrated through the mirror unit 20 adjacent to a center thereof. In detail, one end of the tube 30 is connected to the cover 22 and the opening 301 is penetrated through the glass component 24 of the mirror unit 20 adjacent to a center thereof.

The present invention has characteristics and functions as followed. When a dentist is using the dental mirror, the dental mirror of the present invention can simultaneously suck liquid or fragment in patient's oral cavity. It does not need to alternatively use one mouth mirror and one suction device. Therefore, there are no many pauses during treatment, and the treatment time can be saved, even manpower of assistant can be saved.

According to the present invention, the tube 30 can be detachably assembled to the handle 10, therefore the tube 30 for sucking liquid can be a disposable element, and the tube 30 is replaceable.

According to the present invention, the tube 30 can be detached from the mirror unit 20, so that the tube 30 is extended to a position needed to suck liquid. In addition, the tube 30 also can be attached to the back of the mirror unit 20, and preferably is uniformly distributed on a periphery of the mirror unit 20 to provide an omnidirectional sucking for liquid.

The descriptions illustrated supra set forth simply the preferred embodiments of the present invention; however, the characteristics of the present invention are by no means restricted thereto. All changes, alterations, or modifications conveniently considered by those skilled in the art are deemed to be encompassed within the scope of the present invention delineated by the following claims.

What is claimed is:

1. A dental mirror:
a handle (10);
a mirror unit (20), connected to one end of the handle (10);
a tube (30), disposed at a periphery of the mirror unit (20), wherein the tube (30) is formed with a plurality of inhaling holes (36).

2. The dental mirror as claimed in claim 1, wherein the tube (30) is detachably connected to the handle (10).

3. The dental mirror as claimed in claim 1, wherein the tube (30) has a free end (32) and a fixing end (31), with the inhaling holes (36) arranged between the free end (32) and the fixing end (31); wherein the fixing end (31) is fixedly mounted or detachably connected to the handle (10).

4. The dental mirror as claimed in claim 1, wherein the tube (30) includes at least two fork portions (37), with at least one of the fork portions (37) had a plurality of inhaling holes (36) disposed thereon.

5. The dental mirror as claimed in claim 1, wherein the handle (10) is hollow-shaped, and the tube (30) is communicated with an inner of the handle (10).

6. The dental mirror as claimed in claim 1, wherein one end of the tube (30) has a connecting element (33), the connecting element (33) has an inserting portion (331) and a blocking portion (332) connected to the inserting portion (331), wherein the inserting portion (331) is inserted into the handle (10), the blocking portion (332) is abutted against an outer edge of the handle (10).

7. The dental mirror as claimed in claim 1, wherein the tube (30) is extended from the handle (10) and attached to a back surface of the mirror unit (20), or surrounded in an annular manner and attached to an edge of a back of the mirror unit (20).

8. The dental mirror as claimed in claim 1, wherein the inhaling holes (36) are distributed on an end of the tube (30) or distributed through the tube (30).

9. The dental mirror as claimed in claim 1, wherein the tube (30) is flat-shaped and attached to a back of the mirror unit (20).

10. The dental mirror as claimed in claim 1, wherein the inhaling holes (36) are formed on a back of the mirror unit (20), and the tube (30) has an arc-shaped opening (301), with the arc-shaped opening (301) trimmed to a periphery of the mirror unit (20).

11. The dental mirror as claimed in claim 1, wherein the mirror unit (20) includes a cover (22) and a glass component (24) disposed on the cover (22), wherein the tube (30) has a supporting part (34) extended to the glass component (24), and the supporting part (34) is abutted against the glass component (24).

12. The dental mirror as claimed in claim 1, wherein an end of the tube (30) has an opening (301), and the opening (301) is passed through the mirror unit (20).

13. The dental mirror as claimed in claim 1, wherein the handle (10) is hollow-shaped, and the tube (30) is extended and disposed in the handle (10).

14. The dental mirror as claimed in claim 1, wherein the handle (10) is hollow-shaped, and the tube (30) is either inserted into the handle (10) or fixed to a periphery of the handle (10).
